(19) United States
(12) Patent Application Publication
(10) Pub. No.: US 2006/0129048 A1
(43) Pub. Date: Jun. 15, 2006

(54) Temperature-measuring manometer

(76) Inventors: Kun-Sung Chen, Taipei County (TW); Ying-Chao Lin, Taipei County (TW); Chun-Nan Chen, Taipei County (TW); Hsing Ouyang, Taipei County (TW); Yao Ouyang, Taipei County (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/010,354

(22) Filed: Dec. 14, 2004

(51) Int. Cl. A61B 5/02; A61B 5/00

(52) U.S. Cl. 600/483; 600/490; 600/549

(57) ABSTRACT

A temperature measuring manometer capable of measuring a patient's blood pressure and the temperature in the environment comprises a pressured sensor that transforms physical signals to a blood pressure signal; a temperature module that receives a temperature signal from the environment; an A/D converter coupled to the pressured sensor and the temperature module for receiving the blood pressure signal and the temperature signal so as to transform an analogue signal into a digital signal; a microprocessor coupled to the A/D converter for processing the digital signal of the blood pressure signal and the temperature signal; a clock unit coupled to the microprocessor for transmitting a clock signal to the microprocessor; and a memory unit coupled to the microprocessor for storing the digital data.

### BACKGROUND OF THE INVENTION

1. Field of the Invention

The present invention relates to a temperature-measuring manometer, and more specifically relates to a manometer capable of measuring blood pressure and the temperature of an environment.

2. Description of the Related Art

Blood pressure is measure of the pressure of blood flowing through a blood vessel wall. Moreover blood pressure comprises a systolic pressure and a diastolic pressure. An unusual blood pressure is usually a warning of a serious illness such as apoplexy, heart disease or uremia. Therefore measuring blood pressure assists medical professionals in determining if a patient is suffering from an illness.

A major part of measuring blood pressure is inflating the arteries bag so that it compresses the patient's blood vessels. Using a stethoscope or other inductors also measures blood pressure, may be either a mercury sphygmomanometer (Sphygmomanometer) or an electronic automatic sphygmomanometer (Auscultation Sphygmomanometer). All these devices have in common the need to inflate the arteries bag so as to prevent blood flow. By increasing the pressure against the arteries, blood ceases to flow through the blood vessels so that the patient's blood pressure can then be measured. By decreasing the barometric pressure, the blood may then resume its ordinary pulse and flow. When the first sound of the N.S. Korotkoff sound effect is detected, the device reads the pressure gauge as the Systolic Pressure, then gradually reduces the pressure of the arteries bag, the vascularization channel widens, and the blood resumes its normal smooth flow through the blood vessels, as the arteries bag pressure decreases, the channel gradually opens. When the N.S. Korotkoff sound effect begins to weaken, the last sound is recorded as the Diastolic Pressure.

A Sphygmomanometer user needs to have their blood pressure regularly measured. However, temperature changes in the environment affect blood pressure, thus creating abnormalities. As such, it will be difficult for a doctor to judge whether an abnormality is due to a change in the patient's condition or due to environmental changes. To compensate for this, a sphygmomanometer simultaneously takes an environmental temperature measurement as it measures a patient's blood pressure.

### SUMMARY OF THE INVENTION

Accordingly, the objective of the present invention is to provide a temperature module that measures the temperature of the environment, and, after the user has measured the patient's blood pressure, records and displays the environmental temperature and the patient's blood pressure.

The temperature-measuring manometer of the present invention obtains a patient's blood pressure through a pressured sensor and obtains the temperature of the air through the temperature module. The information is then transmitted to an A/D converter coupled to the pressured sensor and the temperature module. The A/D converter receives the blood pressure signal and the temperature signal and transforms the analogue signal to a digital signal. A microprocessor coupled to the A/D converter for processing the digital signal of the blood pressure signal and the temperature signal outputs the blood pressure data and the temperature data. This data is then transmitted to a memory unit coupled to a microprocessor for storage and transmission to a display unit.

### BRIEF DESCRIPTION OF THE DRAWINGS

The various objectives and advantages of the present invention will be more readily understood from the following detailed description when read in conjunction with the appended drawing, in which:

FIG. 1 is a circuit block diagram of the present invention;

FIG. 2 is a circuit diagram of a pressured sensor of the present invention;

FIG. 3 shows a perspective drawing of the control chip of the present invention;

FIG. 4 shows a perspective drawing of the clock unit of the present invention;

FIG. 5 shows a perspective drawing of the memory unit of the present invention;

FIG. 6 shows a perspective drawing of the temperature module of the present invention.

### DETAILED DESCRIPTION OF THE PREFERRED EMBODIMENTS

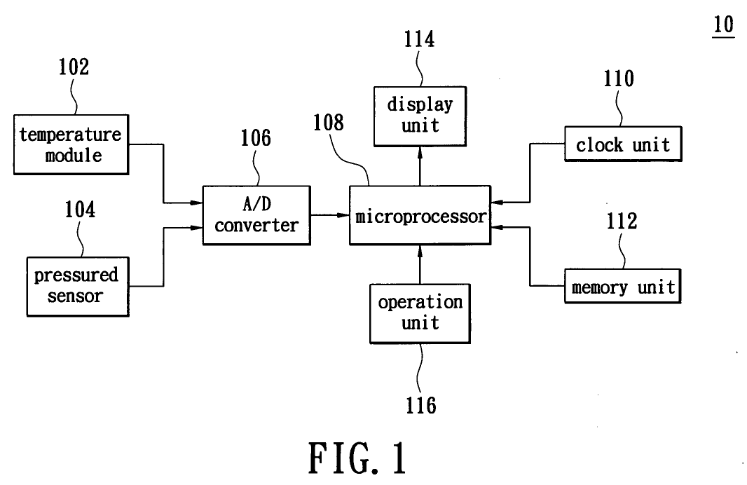

FIG. 1 is a circuit block diagram of the present invention. The manometer 10 of the present invention comprises a temperature module 102, a pressured sensor 104, an A/D converter 106, a microprocessor 108, a clock unit 110, a memory unit 112, a display unit 114 and an operation unit 116.

The manometer 10 of the present invention receives a physical signal in the patient through a CUFF filling and draining the air. The pressured sensor 104 coupled to the CUFF for receiving the physical signal in the patient and transforming the physical signal to an analogue voltage signal so that the analogue voltage signal is a blood pressure signal. Moreover the temperature module 102 receives a physical signal in the environment and transforms the physical signal to an analogue voltage signal so that the analogue voltage signal is a temperature signal. The A/D converter 106 is coupled to the pressured sensor 104 and the temperature module 102 to receive the blood pressure signal and the temperature signal so as to transform the analogue signal of the blood pressure signal and the environmental temperature signal into a digital signal. The microprocessor 108 is coupled to the A/D converter 106 to receive and process the digital signal of the blood pressure signal and the environmental temperature signal so as to output environmental temperature data and blood pressure data. The memory unit 112 is coupled to the microprocessor 108 for storing the environmental temperature data, the blood pressure data and the recorded time data. This data is then transmitted to the display unit 114 for display. In the above-mentioned description, the blood pressure data comprises systolic pressure and diastolic pressure.

Reference is again made to FIG. 1. The clock unit 110 transmits a clock signal to the microprocessor 108, and the clock signal is the reference clock for the microprocessor 108 for processing digital data. In FIG. 1 the user operates an operation unit 11.6, which is coupled to the microprocessor 108 to transmit a control signal to the microprocessor 108 so as to enforce the various functions of the manometer 10 by a patient-machine interface. The operation unit 116 comprises a power button, a memory button, a general mode button etc. The user pushes the power button to turn the device on, pushes the memory button to store the environmental temperature data and the blood pressure data, and pushes the general mode button to set the time and choose between recording the environmental temperature in either Fahrenheit or Celsius.

Reference is again made to FIG. 1. The present invention receives the physical signal from the environment by the temperature module 102 in standby mode. The temperature module 102 transforms the physical signal to the analogue voltage signal, and the analogue voltage signal is transmitted to the A/D converter 106 for transformation into a digital signal. The digital signal is then transmitted to the microprocessor 108 for processing. After processing the digital signal is transmitted to the display unit 114 for display. The user then pushes the general mode button to set the time and choose between recording the environmental temperature in either Fahrenheit or Celsius.

Reference is again made to FIG. 1. The present invention receives the environmental temperature signal, then displays and stores the environmental temperature data, the blood pressure data and the time data after measuring. So the present invention has the functions of measuring the blood pressure and measuring the environmental temperature, storing the time, a patient's blood pressure and the environmental temperature simultaneously for a doctor's reference.

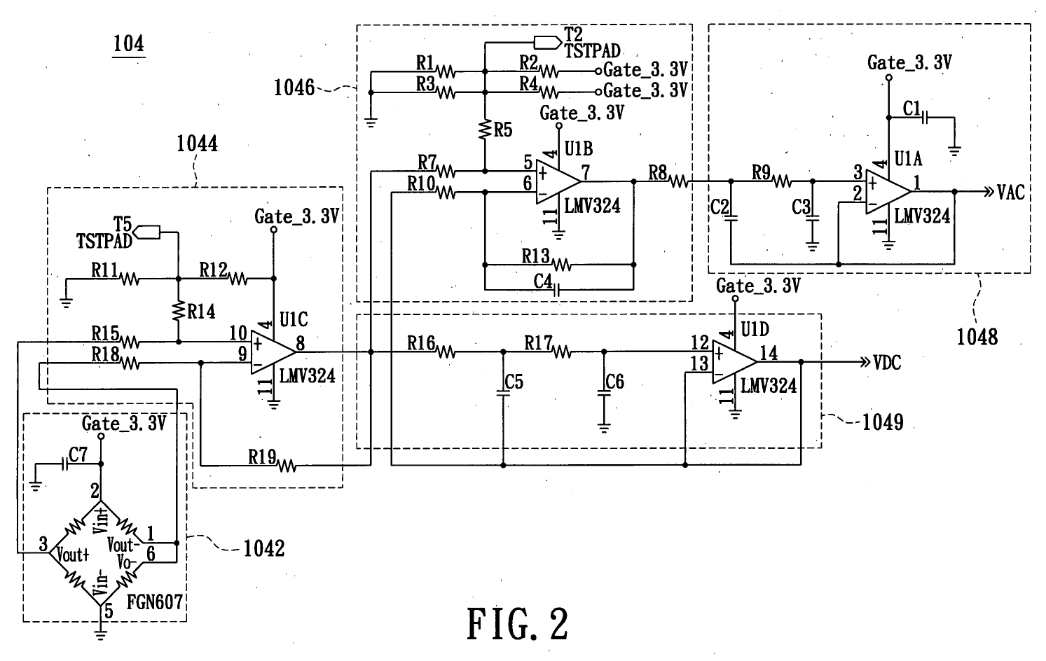

FIG. 2 is a circuit diagram of a pressured sensor 104 of the present invention. When the user puts their arm into the CUFF, the CUFF fills and drains air. At this time, the pressured sensor 104 detects the different pressure signal from the CUFF. A Wheatstone Bridge 1042 receives the different pressure signal and outputs a difference voltage; the difference voltage is related to the pressure signal. A difference amplifier 1044 receives the difference voltage for amplifying the signal. In the first loop, the amplified signal is transmitted to a linear amplifier 1046 for amplification. The second amplified signal is transmitted to a first filter 1048 for filtering. The first filter 1048 outputs a pulse voltage VAC to the A/D converter 106. Moreover, in another loop, the amplified signal is transmitted to a second filter 1049 for filtering. The second filter 1049 outputs a level voltage VDC to the A/D converter 106. The systolic pressure and the diastolic pressure are related to the pulse voltage VAC and the level voltage VDC. In the above-mentioned description, the difference amplifier 1044 consists of an amplifier U1C connected to a plurality of resistors, and the linear amplifier 1046 consists of an amplifier U1B connected to a plurality of capacitors and resistors, and the first filter 1048 consists of an amplifier U1A connected to a plurality of capacitors and resistors.

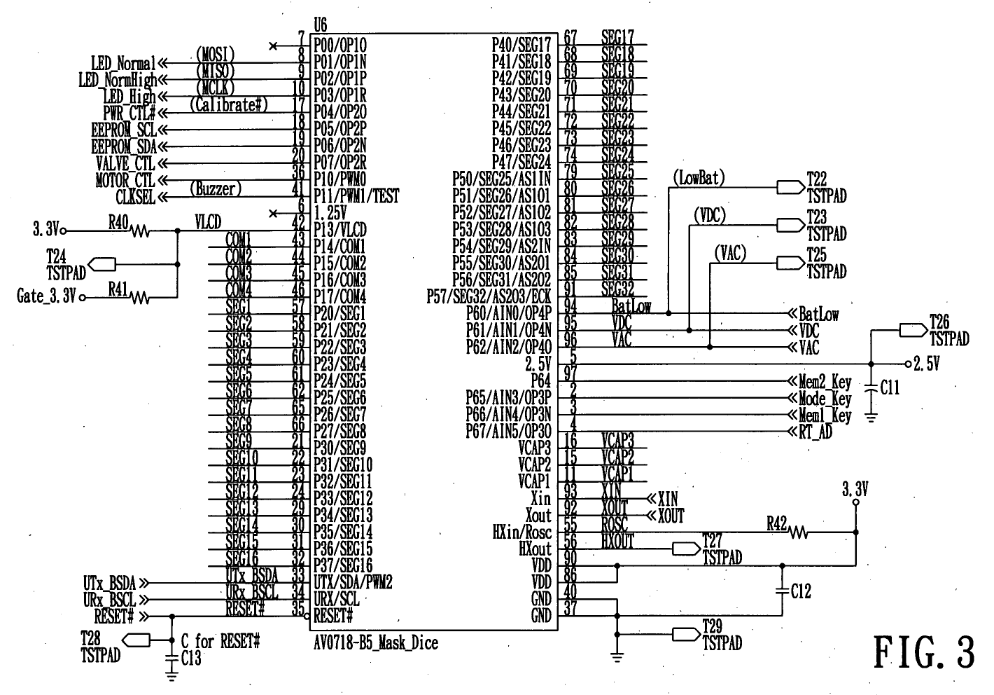

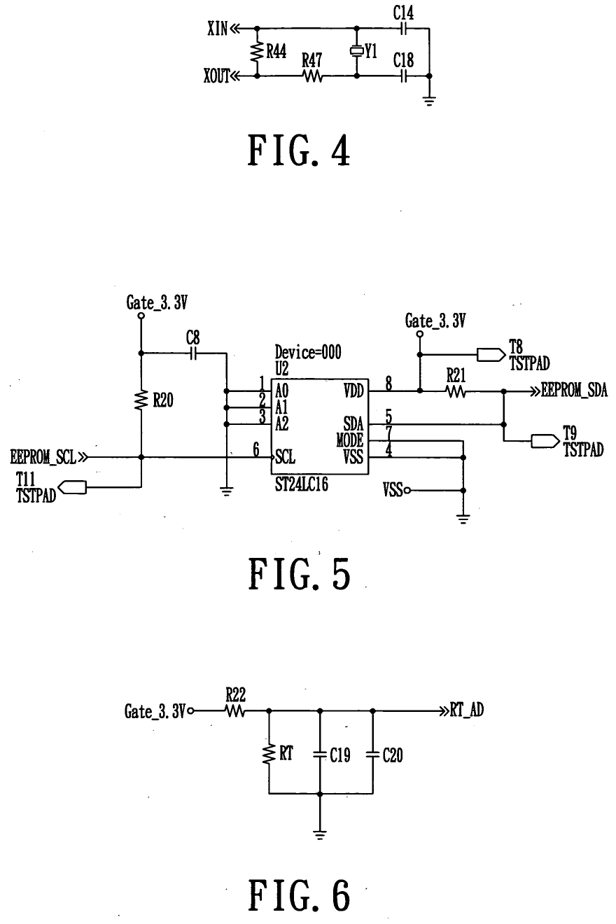

FIG. 3 shows a perspective drawing of the control chip of the present invention. The control chip U6 has the functions of the A/D converter 106 and the microprocessor 108. FIG. 4 shows a perspective drawing of the clock unit of the present invention. The clock unit 110 consists of an oscillator Y1 connected to a plurality of capacitors and resistors. FIG. 5 shows a perspective drawing of the memory unit of the present invention. The memory unit 112 consists of a memory chip U2 connected to a plurality of capacitors and resistors. FIG. 6 shows a perspective drawing of the temperature module of the present invention. The temperature module 102 consists of a thermistor RT connected to a plurality of capacitors and resistors.

Therefore the manometer of the present invention uses a temperature module to obtain a temperature signal from the environment, and, after the user has measured their blood pressure, the temperature signal and the blood pressure signal is recorded and displayed. So the present invention measures both a patient's blood pressure and the environmental temperature, and stores the time, the patient's blood pressure and the environmental temperature simultaneously for a doctor's reference.

Although the present invention has been described with reference to the preferred embodiments thereof, it will be understood that the invention is not limited to the details thereof. Various substitutions and modifications have been suggested in the foregoing description, and other will occur to those of ordinary skill in the art. Therefore, all such substitutions and modifications are intended to be embraced within the scope of the invention as defined in the appended claims.

What is claimed is:

1. A temperature measuring manometer receives a physical signal from a CUFF, comprising:
a pressured sensor transforming the physical signal to a blood pressure signal;
a temperature module receives a temperature signal from the environment;
an A/D converter coupled to the pressured sensor and the temperature module for receiving the blood pressure signal and the temperature signal for transforming an analogue signal to a digital signal;
a microprocessor coupled to the A/D converter for processing the digital signal of the blood pressure signal and the temperature signal;
a clock unit coupled to the microprocessor for transmitting a clock signal to the microprocessor; and
a memory unit coupled to the microprocessor for storing the digital data.

2. The manometer of claim 1, further comprising a display unit coupled to the microprocessor for displaying the information processed by the microprocessor.

3. The manometer of claim 1, further comprising an operation unit coupled to the microprocessor for transmitting a control signal to the microprocessor by a patient-machine interface.

4. The manometer of claim 1, wherein the blood pressure signal is an analogue voltage signal.

5. The manometer of claim 1, wherein the temperature signal is an analogue voltage signal.

6. The manometer of claim 1, wherein the temperature module transforms the physical signal in the environment to an analogue voltage signal.

7. A temperature measuring manometer, comprising:
a pressured sensor that obtains a blood pressure signal;
a temperature module that obtains a temperature signal;
an A/D converter coupled to the pressured sensor and the temperature module for receiving the blood pressure signal and the temperature signal so as to transform an analogue signal into a digital signal; and
a microprocessor coupled to the A/D converter for processing the digital signal of the blood pressure signal and the temperature signal.

8. The manometer of claim 7, further comprising a clock unit coupled to the microprocessor for transmitting a clock signal to the microprocessor.

9. The manometer of claim 7, further comprising a memory unit coupled to the microprocessor for storing the digital data.

10. The manometer of claim 7, further comprising a display unit coupled to the microprocessor for displaying the information processed by the microprocessor.

11. The manometer of claim 7, further comprising an operation unit coupled to the microprocessor for transmitting a control signal to the microprocessor by a patient-machine interface.

12. The manometer of claim 7, wherein the blood pressure signal is an analogue voltage signal.

13. The manometer of claim 7, wherein the temperature signal is an analogue voltage signal.

14. The manometer of claim 7, wherein the pressured sensor transforms the physical signal of a patient's body to an analogue voltage signal.

15. The manometer of claim 7, wherein the temperature module transforms the physical signal in the environment to an analogue voltage signal.
